SEQUENCE LISTING (8 sequences)

SEQ ID NO: 1 - DNA, 1770 bp, Homo sapiens
atgatccctaacatagaaggaatggaaactgaaagtggaaatcaggaaaaggtaatggaagaagaaagcactgaaaagaaaaaagaagttgaaaaaaagaaacggtcacgagttaaacaggtgcttgcagatattgctaagcaagtggacttctggtttggggatgcaaatcttcacaaggatagatttcttcgagaacagatagaaaaatctagagatggatatgttgatatatcactacttgtgtcttttaacaaaatgaaaaaattgactactgatgggaagttaattgccagagcattgagaagttcagctgttgtagagcttgatttggaaggcaccagaatccggaggaaaaaacctctgggggaaagaccaaaggatgaggatgaacgcacagtgtatgtggagttacttcccaaaaatgttaatcacagctggattgaaagagtatttgggaaatgtggcaatgttgtttatataagtataccacattataagtctactggagatccaaagggatttgcgtttgtggaatttgaaacaaaagaacaagcagcaaaagcaattgagtttcttaacaacccaccagaagaagcaccaagaaaacctggcatatttcctaaaacagtgaaaaataagcccattccagccttaagagttgtggaagagaagaaaaagaaaaagaagaagaaaggccgaatgaaaaaggaagacaatatccaagccaaagaagaaaacatggacacaagcaacaccagcatcagtaaaatgaaaagatccagacccacatctgagggctctgacattgagtccactgaaccccaaaagcagtgctcaaagaaaaagaaaaaacgggacagagttgaagcatctagcttacctgaagtcagaacagggaagaggaagagaagcagctctgaagatgcagaatccctagctccccgatcaaaagtaaagaaaattattcagaaagacatcattaaggaagcatcagaagcttccaaggaaaatagagatatagaaatctctactgaagaggaaaaggatactggagatctaaaagatagctctctcttgaaaacaaaaaggaaacataagaaaaaacataaagagagacataaaatgggagaagaagttataccattaagagtgctatcaaagagcgaatggatggatttgaaaaaagagtatttagcgctacaaaaagctagcatggcttctttaaaaaaaacaatatcccaaataaaatcagagtcagaaatggaaacagacagtggagtacctcaaaacactggaatgaaaaatgaaaaaacagccaacagggaagagtgtcgcacccaggagaaagttaatgcaacaggaccacagttcgtgagtggagtgattgtgaagatcattagcacagagcctctacctggcaggaaacaagtccgggatactttggcagcaatctcagaagttctttatgttgatttgctagaaggggatacagaatgccatgctagatttaaaactcctgaggatgctcaagcagtaataaatgcctatacagaaattaacaagaaacactgctggaaactcgagatcctttctggtgatcacgaacaaaggtattggcagaagattttggttgatagacaggcaaaacttaatcagcctcgggaaaagaaaagaggcactgaaaagttaatcaccaaagctgaaaagattagactggcaaagactcaacaagcgagtaaacatataagattttctgaatatgattga

SEQ ID NO: 2 - DNA, 21 bp, Homo sapiens
caggaaatggctgaactagaa

SEQ ID NO: 3 - DNA, 20 bp, Homo sapiens
cagacagugaguaccucatt

SEQ ID NO: 4 - DNA, 21 bp, Homo sapiens
acaagcgaguaaacauauatt

SEQ ID NO: 5 - DNA, 20 bp, Homo sapiens
tggggatgcaaatcttcaca

SEQ ID NO: 6 - DNA, 20 bp, Homo sapiens
ttgctaagcaagtggacttc

SEQ ID NO: 7 - DNA, 5592 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 8 - DNA, 10257 bp, Homo sapiens
atgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagtagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

(19) United States
(12) Patent Application Publication
(10) Pub. No.: US 2021/0087640 A1
(43) Pub. Date: Mar. 25, 2021

(54) APPLICATION OF LARP7 GENE IN CANCER DIAGNOSIS AND TREATMENT

(71) Applicant: SHANGHAI JIAO TONG UNIVERSITY, Shanghai (CN)

(72) Inventor: Bing ZHANG, Shanghai (CN)

(73) Assignee: SHANGHAI JIAO TONG UNIVERSITY, Shanghai (CN)

(21) Appl. No.: 17/054,035

(22) PCT Filed: Mar. 6, 2019
(86) PCT No.: PCT/CN2019/077092

§ 371 Date: Nov. 9, 2020

(30) Foreign Application Priority Data
May 9, 2018 (CN) 201810437660.4

(51) Int. Cl. C12Q 1/6886

(52) U.S. Cl. CPC C12Q 1/6886; C12Q 2600/106; C12Q 2600/158; C12Q 2600/118

(57) ABSTRACT

Applications of the LARP7 gene in preparing a cancer treatment drug, and a drug that increases the sensitivity of cancers to radiotherapy and chemotherapy, and applications of the LARP7 gene in preparing a kit for an early screening or a cancer diagnosis, and in preparing a kit for a prediction of therapeutic efficacy for cancer patients are provided. The LARP7 gene in the cancer treatment drug is used as a target gene, the LARP7 gene in the early-screening/cancer-diagnosis kit is used as a risk assessment marker for a cancer, and the drug increasing the sensitivity of cancers is used for specifically inhibiting a degradation of the LARP7 gene. The cancers include breast cancer, ovarian cancer, cervical cancer, liver cancer, lung cancer, gastric cancer and colorectal cancer.

### CROSS REFERENCE TO THE RELATED APPLICATIONS

This application is the national phase entry of International Application No. PCT/CN2019/077092, filed on Mar. 6, 2019, which is based upon and claims priority to Chinese Patent Application No. 201810437660.4, filed on May 9, 2018, the entire contents of which are incorporated herein by reference.

### TECHNICAL FIELD

The present invention relates to an application of a LARP7 gene in cancer diagnosis and treatment.

### BACKGROUND

Breast cancer is one of the most common malignant tumors in women. It has an incidence rate 7%-10% of all malignant tumors in the human body and shows an increasing trend year by year. The disease occurs frequently in women over 45 years old and is one of the main causes of death among. One in eight women in the United States will develop breast cancer over the course of her lifetime. In recent years, the growth rate of breast cancer incidence in China has been 1% to 2% higher than that in developed countries, and the onset age is younger. According to the breast cancer incidence data of 2009 published by National Cancer Center and the Disease Control and Prevention Bureau of the Ministry of Health of China in 2012, the incidence of breast cancer ranks the first among female malignant tumors in the national cancer registration area in China. The incidence rate (crude rate) of breast cancer in women in China is 42.55 per 100,000 women nationwide, 51.91 per 100,000 women in urban areas, and 23.12 per 100,000 women in rural areas. This shows that breast cancer has become a major public health problem in current society.

Currently available early screening methods for breast cancer mainly include imaging techniques (including mammography, ultrasound and magnetic resonance imaging techniques) and detection of germline mutations in susceptibility genes (mutations in genes such as BRCA1, BRCA2, TP53, CHEK2). However, these available assessment methods are not fully effective in predicting the risk of breast cancer patients for the following reasons. First, only the application value of conventional mammograms in currently available imaging techniques has been confirmed. In the past 20 years, routine mammogram screening has increased the detection rate of early primary breast cancer. This technique has reduced the mortality rate of women aged 50 to 69, and reduced the relative risk of cancer in this group of people by 26%; whereas the value of screening in women aged 40 to 49 by mammography is still controversial.

Second, approximately 54% of patients have no known genetic mutations due to the high heterogeneity of breast cancer, the risk of recurrence and survival time of these patients are difficult to predict using existing markers. Third, although the classification effect of some gene mutations on survival time is statistically significant, the mutation rate of these genes is extremely low, and a majority of patients have no such gene mutations. It is, therefore, impossible to predict the survival time of these patients based on gene mutations. For example, people with mutations in the BRCA1 or BRCA2 genes have a 60-85% probability of developing breast cancer, but the mutation rate of these genes only accounts for 5%-10% of all breast cancer patients. TP53 mutations increase the risk of breast cancer, but the mutation rate of TP53 in breast cancers is only about 1%, which cannot be used for evaluation for most patients. Therefore, it is essential to identify other susceptibility genes of breast cancer and use them as a basis for early screening work to reduce the proportion of patients with advanced breast cancer.

With the improvement of surgical techniques for breast cancer and the combined application of biotherapy and chemotherapy, 90% of patients with primary breast cancer are well cured. However, once recurrence or metastasis occurs, survival rates of the patients drop to less than 10%, with a median survival time of only about three years. Currently, the main treatments for recurrent and metastatic breast cancer are radiotherapy, chemotherapy and biotherapy. The above-mentioned treatments still have their associated limitations, including the following reasons. Adjuvant chemotherapy for breast cancer is relatively common in China, and about 81.4% of patients with invasive breast cancer undergo chemotherapy. Some patients with recurrent breast cancer, however, are resistant to common chemotherapeutic drugs, resulting in poor therapeutic effect. Additionally, although the therapeutic effect on patients is improved to a certain extent by novel chemotherapeutic drugs and drug repurposing (lipid anthracyclines, once-a-week use of taxanes), a variety of therapeutic complications, including bone marrow suppression, nausea, vomiting, hair loss, neurotoxicity, and dermal toxicity can result.

Still another limitation of current radiotherapy, chemotherapy and biotherapy treatments for breast cancer involves the latest development is targeted therapy for HER2. Randomized clinical trials have shown that anti-HER2 (trastuzumab, also called Herceptin) targeted therapy combined with chemotherapy was superior to chemotherapy alone, and improved the survival rate 1 year, treatment remission rate and disease progression time for patients. HER-2/neu protein overexpression is only detected in 25-30% of human breast cancers, however, and the drug is not included in medical insurance. This results in substantial out-of-pocket payment of patients, which limits the application of the drug. According to statistics, only 40% of metastatic patients in China receive second-line treatment, and only one-quarter receive third-line treatment. Therefore, finding new therapeutic targets and timely assessing recurrence risk and survival time for patients are conducive to making up for the limitations of existing treatments and providing patients with accurate treatment.

In conclusion, screening new breast cancer susceptibility genes will help to make up for the deficiencies of existing assessment standards, achieve more accurate risk evaluation, contribute to develop effective targeted therapeutic drugs, and promote the development of precision medicine for breast cancer.

### SUMMARY

The objective of the present invention is to provide an application of a LARP7 gene in cancer diagnosis and treatment. In the present invention, it is discovered for the first time that the expression level of the LARP7 gene is directly related to the occurrence of cancers (including breast cancer, ovarian cancer, cervical cancer, liver cancer, lung cancer, and gastric cancer) and the sensitivity of patients to radiotherapy and chemotherapy, revealing a new mechanism of the LARP7 promoting tumorigenesis and affecting the treatment and prognosis of tumor patients, and the LARP7 gene can be used for the development of anti-tumor drugs and diagnostic kits as well as related preparations.

The objective of the present invention is achieved through the technical solutions as follows.

In the first aspect, the present invention relates to an application of a LARP7 gene (as shown in SEQ ID NO.1) in preparing a drug for treating a cancer.

Preferably, the LARP7 gene in the drug development is used as a target gene.

Preferably, the drug is used for inhibiting an expression level of the LARP7 gene.

Preferably, the cancer is breast cancer, ovarian cancer, cervical cancer, liver cancer, lung cancer or gastric cancer.

Preferably, the cancer is breast cancer, ovarian cancer, cervical cancer, liver cancer, lung cancer, gastric cancer or colorectal cancer.

In the second aspect, the present invention relates to an application of a LARP7 gene (as shown in SEQ ID NO.1) in preparing a kit for an early screening or a cancer diagnosis, and the LARP7 gene is used as a risk assessment marker for a cancer.

Preferably, the cancer is breast cancer, ovarian cancer, cervical cancer, liver cancer, lung cancer or gastric cancer.

Preferably, the cancer is breast cancer, ovarian cancer, cervical cancer, liver cancer, lung cancer, gastric cancer or colorectal cancer.

In the third aspect, the present invention relates to an application of a LARP7 gene (as shown in SEQ ID NO.1) in preparing a kit for a prediction of therapeutic efficacy for cancer patients.

Preferably, the prediction of the therapeutic efficacy includes a prediction of a prognostic survival time and a recurrence situation.

Preferably, the cancer is breast cancer, ovarian cancer, cervical cancer, liver cancer, lung cancer or gastric cancer.

Preferably, the cancer is breast cancer, ovarian cancer, cervical cancer, liver cancer, lung cancer, gastric cancer or colorectal cancer.

In the fourth aspect, the present invention relates to an application of a LARP7 gene (as shown in SEQ ID NO.1) in preparing a drug for increasing a sensitivity of a cancer to radiotherapy and chemotherapy, and the drug is used for specifically inhibiting a degradation of the LARP7 gene.

Preferably, the drug is used to improve an overall survival time or a relapse-free survival time.

Preferably, the cancer is breast cancer, ovarian cancer, cervical cancer, liver cancer, lung cancer or gastric cancer.

Preferably, the cancer is breast cancer, ovarian cancer, cervical cancer, liver cancer, lung cancer, gastric cancer or colorectal cancer.

The applicant of the present invention has found that the increased expression of LARP7 protein in cancer tissues is related to the tumorigenesis and prognosis of tumors, and is closely related to the occurrence and the sensitivity of radiotherapy and chemotherapy of cancers, and the LARP7 protein can be used for the development of targeted drugs and prognostic and diagnostic kits for cancer treatments (such as breast cancer). Compared with the prior art, the present invention has the advantages as follows.

Previously, the prognostic markers only focused on genome-level changes, such as chromatin structural variations and gene mutations, but rarely analyzed and verified the correlation between gene expression and prognosis of breast cancer patients from the protein-level. However, most breast cancer patients lack mutations in related genes and cannot be assessed for risk through existing tumor markers. In view of the shortcomings of previous studies, the present invention adopts a combination of bioinformatics and experiments, from the three levels of cells, animals, and clinical samples, to prove that the RNA-binding protein LARP7 has a strong prediction effect on the tumorigenesis, the prognostic survival time and recurrence situation of patients, and prove that the LARP7 can be used as a risk assessment marker for cancers (such as breast cancer) for early screening and prediction of therapeutic efficacy of cancer patients. The reduction of LARP7 expression can inhibit tumor proliferation, so the development of drugs that inhibit LARP7 expression may achieve therapeutic effects on tumors including breast cancer. Conversely, the increase of LARP7 expression can increase the sensitivity to first-line chemotherapeutic drugs and radiotherapy. Screening of drugs that specifically inhibit the degradation of LARP7 protein will be beneficial to increase the sensitivity of the tumor to radiotherapy and chemotherapy and improve the overall survival time or relapse-free survival time of the patients.

### BRIEF DESCRIPTION OF THE DRAWINGS

Other features, objectives, and advantages of the present invention will become more apparent by reading a detailed description of the non-limiting embodiment with reference to the drawings below.

FIG. 1A is a schematic diagram showing expression levels of LARP7 in a paratumor tissue and breast cancer cells of stage I to IV by immunohistochemistry (IHC) analysis;

FIG. 1B is a diagram showing immunohistochemistry staining results of 210 breast cancer patients through statistical analysis; according to the expression levels of LARP7, tissue samples are divided into three categories: strong, intermediate and weak; the expression levels of LARP7 in cancer tissues of stage I to IV are higher than that in the paratumor tissue through Wilcoxon rank-sum test, and the expression levels of LARP7 in stages III and IV are also higher than that in stage I;

FIG. 2A is a diagram showing proliferation curves of MDA-MB-231 xenograft tumor, indicating that LARP7 overexpression promotes tumor growth;

FIG. 2B is a diagram showing sizes and metastasis situations of the xenograft tumor by luciferase assay;

FIG. 2C is a diagram showing statistical results of sizes of isolated tumors, indicating a significant increase in tumor size in the LARP7 overexpression group (LARP7^{OE});

FIG. 2D is a diagram showing proliferation curves of HCC1937 breast cancer xenograft tumor;

FIG. 2E is a diagram showing final sizes of the tumors by luciferase assay;

FIG. 2F is a diagram showing statistical results of final sizes of the isolated tumors;

FIG. 3A is a schematic diagram showing that knocking down LARP7 in triple-negative breast cancer cells HCC1937 increases viability of the cells after treated with CDDP (cisplatin) by CCK8 (cell counting kit-8) cell viability experiments;

FIG. 3B is schematic diagram showing knocking down LARP7 in triple-negative breast cancer cells HCC1937 increases viability of the cells after treated with MMS (mitomycin) by CCK8 cell viability experiments;

FIG. 3C is schematic diagram showing knocking down LARP7 in triple-negative breast cancer cells MDA-MB-231 increases viability of the cells after treated with γ-ray (IR) by CCK8 cell viability experiments;

FIG. 3D is schematic diagram showing knocking down LARP7 in triple-negative breast cancer cells HCC1937 increases viability of the cells after treated with γ-ray by CCK8 cell viability experiments;

FIG. 3E is schematic diagram showing LARP7 overexpression increases the sensitivity of MDA-MB-231 to CDDP;

FIG. 3F is schematic diagram showing LARP7 overexpression increases the sensitivity of MDA-MB-231 to MMS;

FIG. 3G is schematic diagram showing LARP7 overexpression increases the sensitivity of MDA-MB-231 to γ-ray;

FIG. 3H is schematic diagram showing LARP7 overexpression increases the sensitivity of HCC1937 to γ-ray;

FIG. 3I is schematic diagram showing overexpressing LARP7 in MDA-MB-231 significantly increases the sensitivity of the cells to IR by clone formation experiments;

FIG. 3J is schematic diagram showing overexpressing LARP7 in MDA-MB-231 significantly increases the sensitivity of the cells to CDDP by clone formation experiments;

FIG. 4A is a schematic diagram showing LARP7 overexpression in cervical cancer cells Hela increases cell sensitivity of the cells treated with CDDP by CCK8 cell viability experiments;

FIG. 4B is a schematic diagram showing LARP7 overexpression in Hela increases cell sensitivity of the cells treated with MMS by CCK8 cell viability experiments;

FIG. 4C is a schematic diagram showing LARP7 overexpression in Hela increases cell sensitivity of the cells treated with X-ray by CCK8 cell viability experiments;

FIG. 4D is a schematic diagram showing knocking down LARP7 increases resistance of the Hela to CDDP;

FIG. 4E is a schematic diagram showing knocking down LARP7 increases resistance of the Hela to X-ray;

FIG. 4F is a schematic diagram showing LARP7 overexpression in the Hela significantly increases cell sensitivity of the cells to CDDP by clone formation experiments;

FIG. 4G is a schematic diagram showing LARP7 overexpression in the Hela significantly increases cell sensitivity of the cells to IR by clone formation experiments;

FIG. 5A is a schematic diagram showing expression levels of LARP7 in recurrent breast cancer patients and non-recurrent patients in a data set of Cancer Cell 2004/06/01;

FIG. 5B is a schematic diagram showing expression levels of LARP7 in recurrent breast cancer patients and non-recurrent patients in a data set of Nature Medicine 2008/05/01;

FIG. 5C is a schematic diagram showing expression levels of LARP7 in recurrent breast cancer patients and non-recurrent patients in a data set of N Engl J Med 2002/12/19;

FIG. 5D is a schematic diagram showing expression levels of LARP7 in recurrent breast cancer patients and non-recurrent patients in a data set of Breast Cancer Res Treat 2012/04/01;

FIG. 6A is a schematic diagram showing impacts of LARP7 on relapse-free survival (RFS) times of breast cancer assessed by Kaplan-Meier survival analysis; gray lines in the figure indicate samples with high expression of LARP7, and black lines indicate samples with low expression of the LARP7 genes;

FIG. 6B is a schematic diagram showing impacts of BRCA1 on RFS times of breast cancer assessed by Kaplan-Meier survival analysis; gray lines in the figure indicate samples with high expression of BRCA1, and black lines indicate samples with low expression of the BRCA1 genes;

FIG. 6C is a schematic diagram showing impacts of BRCA2 on RFS times of breast cancer assessed by Kaplan-Meier survival analysis; gray lines in the figure indicate samples with high expression of BRCA2, and black lines indicate samples with low expression of the BRCA2 genes;

FIG. 7A is a schematic diagram showing impacts of expression levels of LARP7 on overall survival times of patients with breast cancer assessed by Kaplan-Meier survival analysis;

FIG. 7B is a schematic diagram showing impacts of expression levels of LARP7 on overall survival times of patients with liver cancer assessed by Kaplan-Meier survival analysis;

FIG. 7C is a schematic diagram showing impacts of expression levels of LARP7 on overall survival times of patients with gastric cancer assessed by Kaplan-Meier survival analysis;

FIG. 7D is a schematic diagram showing impacts of expression levels of LARP7 on overall survival times of patients with lung cancer assessed by Kaplan-Meier survival analysis;

FIG. 7E is a schematic diagram showing impacts of expression levels of LARP7 on overall survival times of patients with ovarian cancer assessed by Kaplan-Meier survival analysis;

FIG. 8A is a diagram showing a strategy of radiotherapy and chemotherapy for xenograft tumor mice;

FIG. 8B is a diagram showing knocking out LARP7 in HCC1937 cells reduces the sensitivity of HCC1937 to radiotherapy and CDDP; and

FIG. 8C is a diagram showing LARP7 overexpression in MDA-MB-231 cells increases the sensitivity of MDA-MB-231 to radiotherapy and CDDP.

### DETAILED DESCRIPTION OF THE EMBODIMENTS

The present invention is described in detail below in combination with specific embodiments. The following embodiments are intended to help those skilled in the art to further understand the present invention, rather than limit the present invention in any form. It should be noted that several modifications and improvements may be made by those of ordinary skill in the art without departing from the concepts of the present invention, and such modifications and improvements shall also be considered to be within the protective scope of the present invention.

### Embodiment

The present embodiment provides a genetic marker that can be used for breast cancer risk assessment, and provides a new target for treating breast cancer. The specific findings include the following points:

1) the expression levels of LARP7 in breast cancer tissues and paratumor tissues is significantly different;

2) modulation of LARP7 expression can change the tumorigenic ability of xenograft tumors in mice;

3) modulation of LARP7 expression can change the sensitivity of breast cancer cells to chemotherapeutic drugs and radiotherapy (including γ rays); and

4) the expression values of LARP7 have a significant prediction effect on the survival times of breast cancer patients.

1. High Expression of LARP7 Promotes the Tumorigenesis and Metastasis of Breast Cancer.

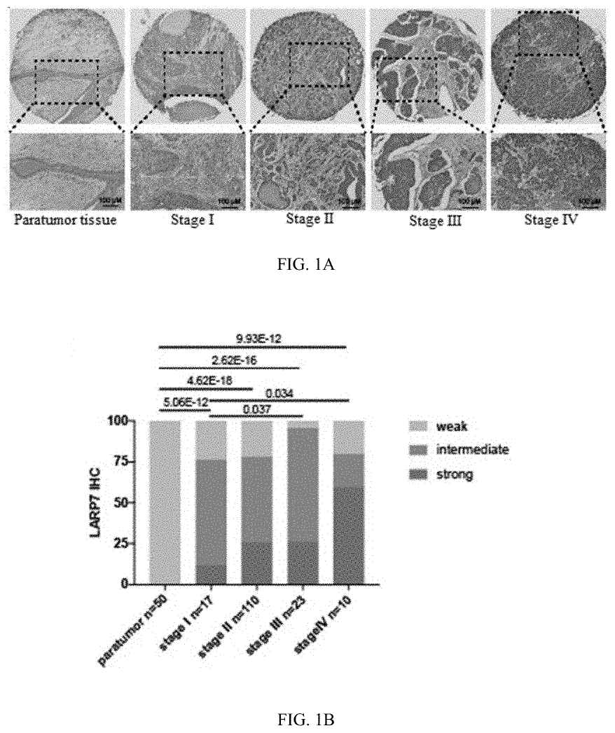

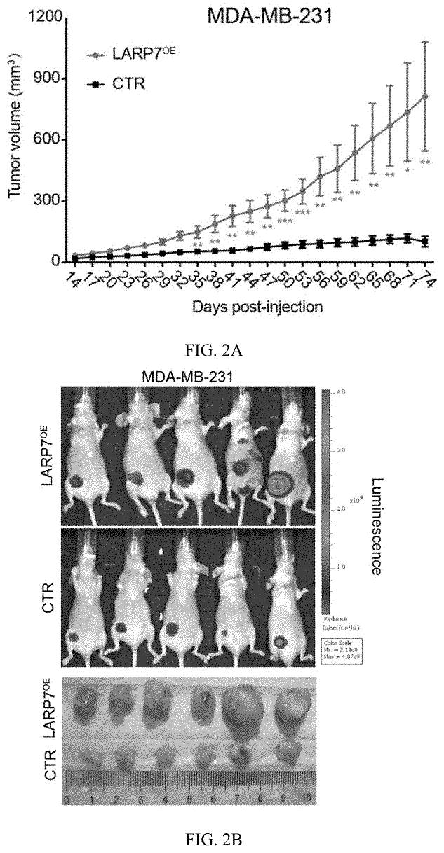

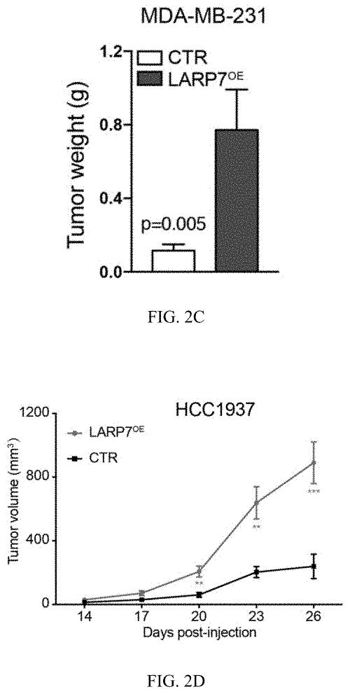

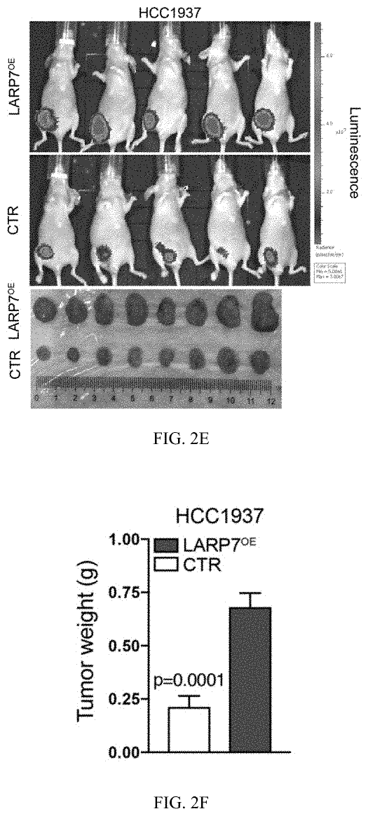

Through immunohistochemistry (IHC) analysis of tissue chips of 210 breast cancer patients, it was found that the expression levels of LARP7 in breast cancer cells of stage I to IV was significantly higher than that of the paratumor tissue (the P values of Wilcoxon rank-sum test is 5.06E-12, 4.62E-18, 2.62E-16, and 9.93E-12, respectively). Moreover, with the increase of tumor malignancy, the expression levels of LARP7 also significantly increased (FIGS. 1A-B). The xenograft experiments in mice showed that overexpression of LARP7 significantly improved the tumorigenic ability of triple-negative breast cancer cells MDA-MB-231 (FIGS. 2A-2C) and HCC1937 (FIGS. 2D-2E) (the tumor volume and tumor weight of mice in the LARP7 overexpression group (LARP7^{OE}) were significantly higher than those in the control group), and promoted the occurrence of metastasis.

2. LARP7 Increases the Sensitivity of Breast Cancer Cells to the Chemotherapeutic Drugs CDDP, MMS and γ-rays.

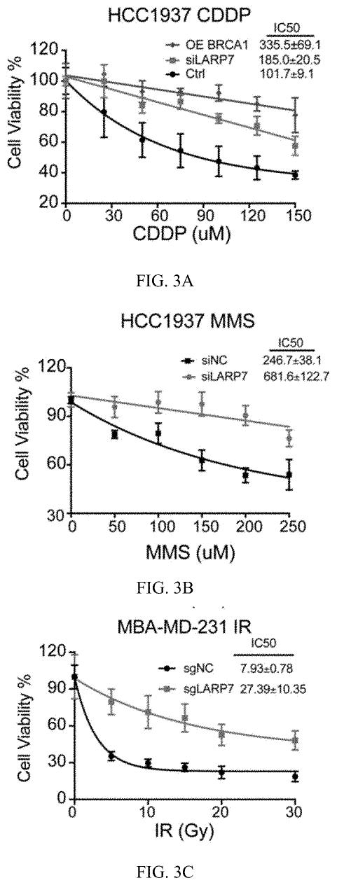

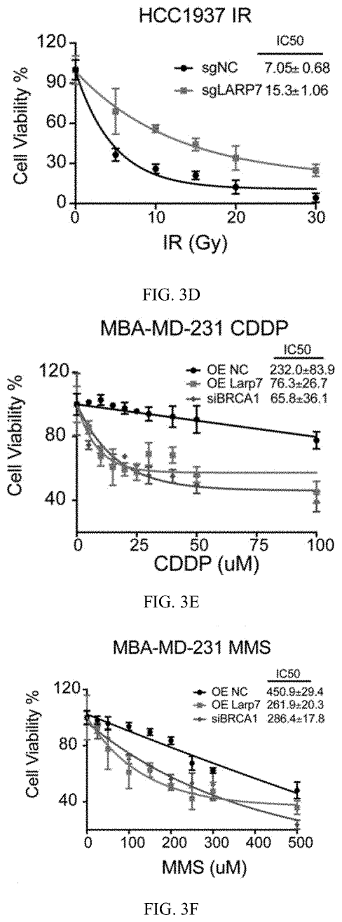

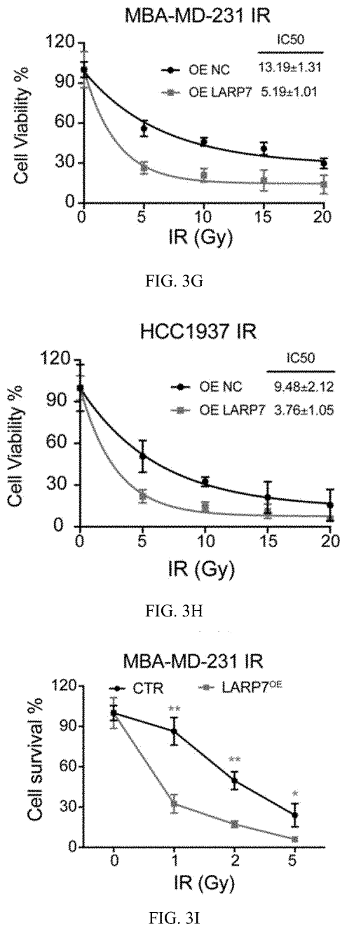

Cell viability experiments measured by CCK-8 (CCK8, Cell Counting Kit-8) have found that LARP7 knockdown resulted in anti-chemotherapeutic drugs and γ-ray resistance in the triple-negative breast cancer cells MDA-MB-231 (TP53 mutation, BRCA1 wild-type cell line) and HCC1937 (TP53 and BRCA1 double mutation). The IC₅₀ (the concentration of drug required for 50% inhibition) of the two types of cells on CDDP, MMS and IR are as follows: chemotherapeutic drugs CDDP (IC₅₀: LARP7-knock-down HCC1937 cells vs. control group=185.0±20.5 μM vs. 101.7±9.1 μM), MMS (IC₅₀: LARP7-knock-down HCC1937 cells vs. control group=681.6±122.7 μM vs. 246.7±38.1 μM), and γ-rays (IC₅₀: LARP7-knock-down MDA-MB-231 cells vs. control group=27.39±10.35 Gy vs. 7.93±0.78 Gy, LARP7-knock-down HCC1937 cells vs. control group=15.3±1.06 Gy vs. 7.05±0.68 Gy) (FIGS. 3A-3D). Conversely, overexpression of LARP7 significantly increased the sensitivity of cells to CDDP (IC₅₀: LARP7-overexpressing MDA-MB-231 cells vs. control group=76.3±26.7 μM vs. 232.0±83.9 μM), MMS (IC₅₀: LARP7-overexpressing MDA-MB-231 cells vs. control group=261.9±20.3 μM vs. 450.9±29.4 μM) and γ-rays (IC₅₀: LARP7-overexpressing MDA-MB-231 cells vs. control group=5.19±1.01 Gy vs. 13.19±1.31 Gy, LARP7-overexpressing HCC1937 cells vs. control group=3.76±1.05 Gy vs. 9.48±2.12 Gy) (FIGS. 3E-3H, and the sequence of siBRCA1 shown in FIG. 3E and FIG. 3F is shown as SED ID NO.2). The clone formation experiment of MDA-MB-231 also further proved that LARP7 increased the sensitivity of cells to DNA toxic reagents (FIGS. 3I-3J).

3. LARP7 Increases the Sensitivity of Cervical Cancer Cells to Chemotherapeutic Drugs CDDP, MMS and X-Rays.

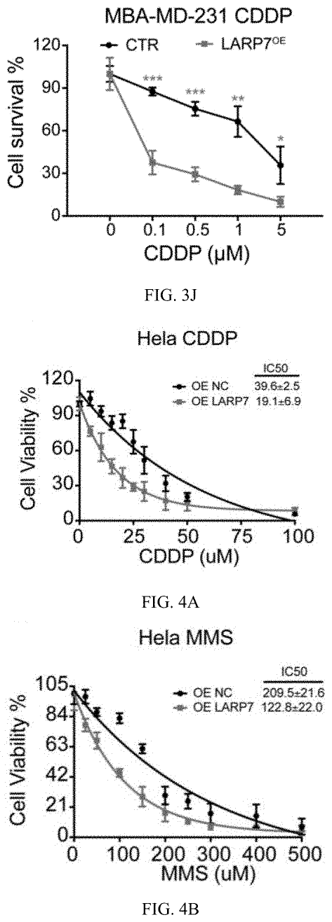

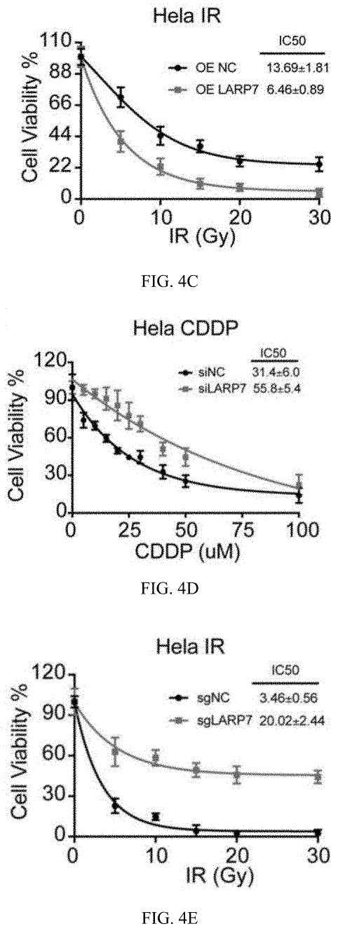

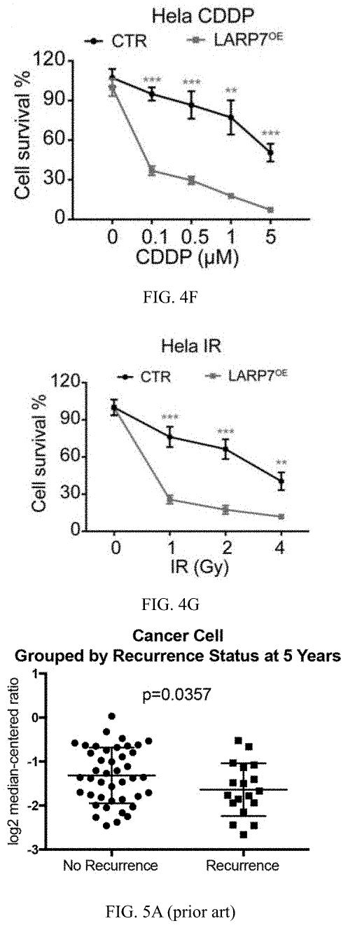

Cell viability experiments measured by CCK-8 (CCK8) have found that overexpression of LARP7 increased the sensitivity of Hela cells to chemotherapeutic drugs CDDP, MMS and IR. The IC₅₀ of Hela cells on CDDP, MMS and IR are as follows: chemotherapeutic drugs CDDP (LARP7 overexpression vs. control group=19.1±6.9 μM vs. 39.6±2.5 μM), MMS (LARP7 overexpression vs. control group=122.8±22.0 μM vs. 209.5±21.6 μM), X-rays (LARP7 overexpression vs. control group=6.46±0.89 μM vs. 13.69±1.81 μM) (FIGS. 4A-4C). Conversely, knocking down LARP7 using a siLARP7 (as shown in SEQ ID NO.3 and SEQ ID NO.4) and a sgLARP7 (as shown in SEQ ID NO.5 and SEQ ID NO.6) significantly increased the resistance of Hela cells to CDDP (IC₅₀: LARP7-knockdown vs. control group=55.8±5.4 μM vs. 31.4±6.0 μM) and X-rays (IC₅₀: LARP7-knockdown vs. control group=20.02±2.44 Gy vs. 3.46±0.56 Gy) (FIGS. 4D-4E). Hela clone formation experiment further demonstrated that LARP7 increased the sensitivity of cells to DNA toxic reagents (FIGS. 4F-4G).

4. LARP7 Promotes the Sensitivity of In Vivo Tumor Tissues to X-Rays and CDDP.

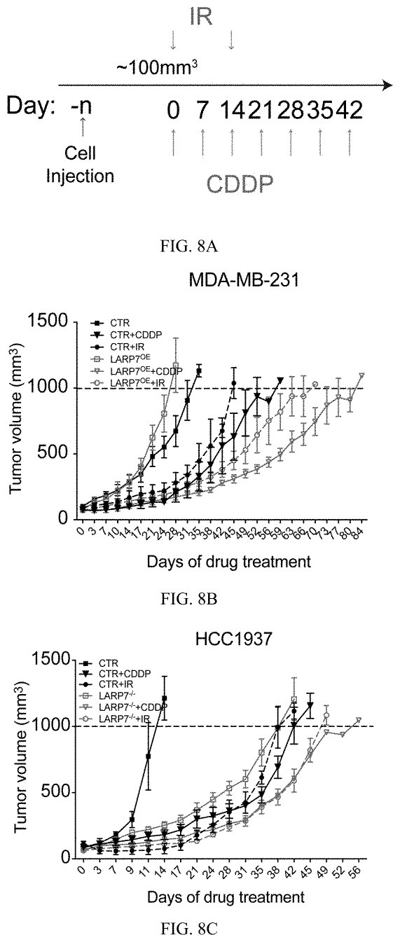

HCC1937 cells showed high expression of LARP7 and were sensitive to CDDP and radiation. However, LARP7-knock-out HCC1937 cells using a sgLARP7 (as shown in SEQ ID NO.5 and SEQ ID NO.6) were less sensitive to CDDP and radiation, resulting in treatment tolerance (FIGS. 8A-8B). Conversely, the expression levels of LARP7 in MDA-MB-231 cells was low, and the MDA-MB-231 cells were tolerant to CDDP and radiation. Artificial overexpression of LARP7 significantly improved the sensitivity of MDA-MB-231 cells to CDDP and radiation. Tumor growth was significantly slowed down after radiotherapy and chemotherapy (FIG. 8C).

5. The Relapse-Free Survival Times of Patients with High LARP7 Expression were Significantly Longer than Those with Low LARP7 Expression.

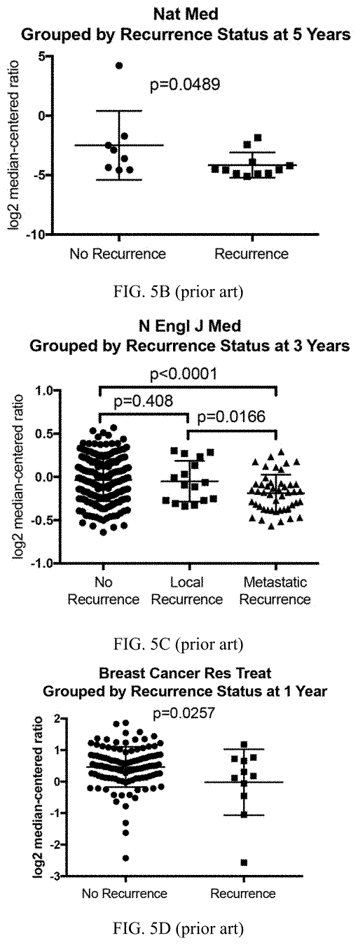

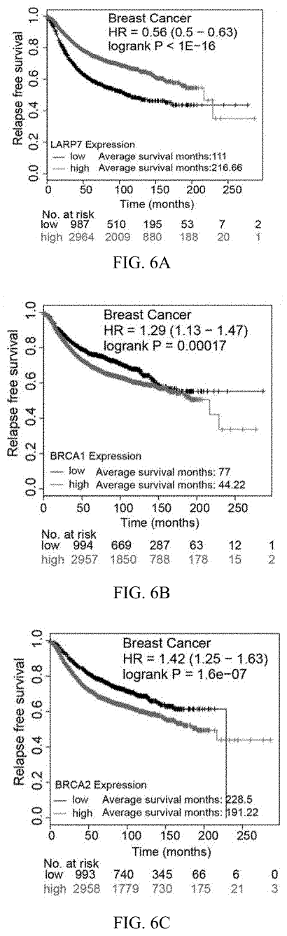

By analyzing four independent data sets (Cancer Cell 2004/06/01, Nature Medicine 2008/05/01, N Engl J Med 2002/12/19 and Breast Cancer Res Treat 2012/04/01), it is found that the expression levels of LARP7 reduced in recurrent breast cancer patients (FIGS. 5A-D), which indicated that patients with low LARP7 expression were very likely to have a poor prognosis. In order to further verify the impact of LARP7 on the prognosis of breast cancer patients, the survival times of 3951 breast cancer patients with receiving chemotherapy were analyzed. The patients were ranked according to the expression values of LARP7 from high to low, and the relapse-free survival times of the patients with the expression values ranked in the top 75% were significantly longer than that of the rest of the patients [Logrank P=1E-16; and homology recombination (HR)=0.56 (0.5-0.63)]. The average relapse-free survival time was 216.66 months in the LARP7 high expression group and 111 months in the LARP7 low expression group (FIG. 6A). Based on the same grouping method, the expression levels and prognosis of the known susceptibility genes BRCA1 (as shown in SEQ ID NO.7) and BRCA2 (as shown in SEQ ID NO.8) were analyzed (FIGS. 6B-6C). The expression levels of BRCA1 and BRCA2 has a significantly lower effect on the prognosis of patients than LARP7 [Logrank P is 0.00017 and 1.6E-7, respectively; and HR is 1.29 (1.13-1.47) and 1.42 (1.25-1.63), respectively]. The average relapse-free survival time was 44.22 months in the BRCA1 high expression group, 77 months in the BRCA1 low expression group, 191.21 months in the BRCA2 high expression group, and 228.85 months in the BRCA2 low expression group.

6. The Overall Survival Times of Patients with High LARP7 Expression were Significantly Longer than that of Patients with Low LARP7 Expression.

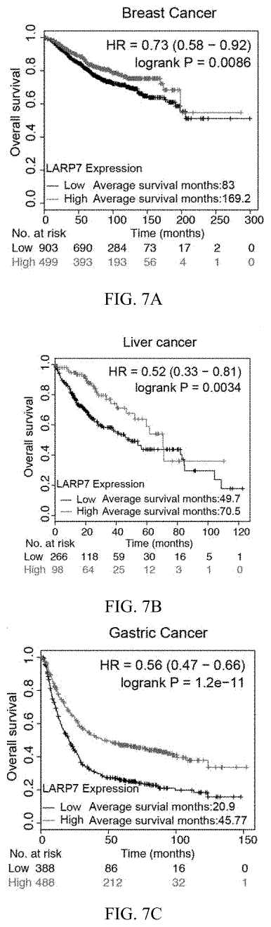

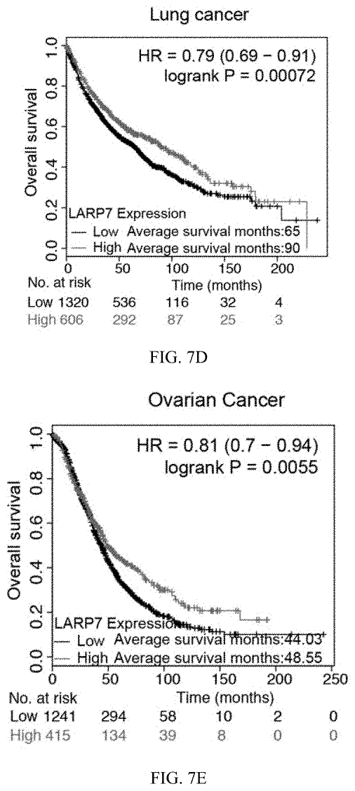

Subsequently, among 1402 breast cancer patients (FIG. 7A), 364 liver cancer patients (FIG. 7B), 876 gastric cancer patients (FIG. 7C), 1926 lung cancer patients (FIG. 7D) and 1656 ovarian cancer patients (FIG. 7E), the relationship between the expression levels of LARP7 and the overall survival times of the patients was analyzed. As shown in FIGS. 6A-C, the prognosis of patients in the LARP7 high expression group was significantly better than that in the LARP7 low expression group (Logrank P values in the above-mentioned five cancers were 0.0086, 0.0034, 1.2E-11, 0.00072, and 0.0055, respectively; and HR was 0.73, 0.52, 0.56, 0.79, and 0.81, respectively).

The specific embodiments of the present invention are described above. It should be understood that the present invention is not limited to the above specific embodiments, and various deformations or modifications within the scope of the claims may be made by those skilled in the art, which shall not affect the substantive content of the present invention.

What is claimed is:

1. A method of using a LARP7 gene in preparing a drug for treating a cancer.

2. The method according to claim 1, wherein the LARP7 gene in the drug is used as a target gene.

3. The method according to claim 1, wherein the cancer is one selected from the group consisting of breast cancer, ovarian cancer, cervical cancer, liver cancer, lung cancer and gastric cancer.

4. The method according to claim 1, wherein the cancer is one selected from the group consisting of breast cancer, ovarian cancer, cervical cancer, liver cancer, lung cancer, gastric cancer and colorectal cancer.

5. A method of using a LARP7 gene in preparing a kit for an early screening or a cancer diagnosis, wherein the LARP7 gene is used as a risk assessment marker for a cancer.

6. The method according to claim 5, wherein the cancer is one selected from the group consisting of breast cancer, ovarian cancer, cervical cancer, liver cancer, lung cancer and gastric cancer.

7. The method according to claim 5, wherein the cancer is one selected from the group consisting of breast cancer, ovarian cancer, cervical cancer, liver cancer, lung cancer, gastric cancer and colorectal cancer.

8. A method of using a LARP7 gene in preparing a kit for a prediction of therapeutic efficacy for patients with a cancer.

9. The method according to claim 8, wherein the prediction of the therapeutic efficacy comprises a prognostic survival time prediction and a recurrence situation prediction.

10. The method according to claim 8, wherein the cancer is one selected from the group consisting of breast cancer, ovarian cancer, cervical cancer, liver cancer, lung cancer and gastric cancer.

11. The method according to claim 8, wherein the cancer is one selected from the group consisting of breast cancer, ovarian cancer, cervical cancer, liver cancer, lung cancer, gastric cancer and colorectal cancer.

12. A method of using a LARP7 gene in preparing a drug for increasing a sensitivity of a cancer to radiotherapy and chemotherapy, wherein the drug is used for specifically inhibiting a degradation of the LARP7 gene.

13. The method according to claim 12, wherein the cancer is one selected from the group consisting of breast cancer, ovarian cancer, cervical cancer, liver cancer, lung cancer and gastric cancer.

14. The method according to claim 12, wherein the cancer is one selected from the group consisting of breast cancer, ovarian cancer, cervical cancer, liver cancer, lung cancer, gastric cancer and colorectal cancer.
